(19) United States
(12) Patent Application Publication
(10) Pub. No.: US 2005/0277626 A1
(43) Pub. Date: Dec. 15, 2005

(54) Methods and compositions for treatment of nicotine dependence and dementias

(75) Inventors: Timothy Dinan, Cobh (IE); Peter Daly, Blackrock (IE)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NeuroCure Ltd., Dublin (IE)

(21) Appl. No.: 11/140,545

(22) Filed: May 27, 2005

Related U.S. Application Data
(60) Provisional application No. 60/574,593, filed on May 27, 2004.
(60) Provisional application No. 60/648,558, filed on Jan. 31, 2005.

(51) Int. Cl. A61K 31/55; A61K 31/5415

(52) U.S. Cl. 514/214.03; 514/225.5; 514/225.8

(57) ABSTRACT

In accordance with the present invention, it has been unexpectedly found that the administration of an acetylcholinesterase inhibitor in combination with a tricyclic antidepressant having anti-muscarinic properties provides a highly effective and well tolerated treatment for nicotine dependence and dementias, such as Alzheimer's disease (AD). In one aspect, it is effective in the treatment of nicotine dependence, as well as in the treatment and mitigation of nicotine withdrawal symptoms and in the effectuation of smoking cessation. In another aspect, it is effect in the treatment and mitigation of dementias such as Alzheimer's disease.

### CROSS-REFERENCES TO RELATED APPLICATIONS

The present application claims the benefit under 35 U.S.C. § 119(e) of U.S. Provisional Application No. 60/574,593, filed May 27, 2004, and U.S. Provisional Application No. 60/648,558 filed Jan. 31, 2005, the contents of which are incorporated herein by reference.

### BACKGROUND OF THE INVENTION

The present invention relates to pharmaceutical compositions and methods. More particularly, the invention relates to pharmaceutical compositions and methods for the treatment and mitigation of nicotine dependence.

Currently marketed approaches for the treatment of nicotine dependence include various over the counter approaches for delivery of nicotine, including transdermal patches, chewing gums, etc., which while better than placebo still fail to work in the great majority of smokers. An approved prescription only anti-smoking medication in the US is buproprion (Zyban™). However, buproprion has a number of worrying side effects including the potential for seizures.

Acetylcholine (ACh) was the first neurotransmitter to be discovered when Loewi in 1921 demonstrated that stimulation of the vagus nerve released a chemical which slowed the frog heart. Since then the role of ACh both in the periphery and in the brain has been extensively studied. The key enzymes regulating the synthesis and metabolism of ACh are choline acetyltransferase and acetylcholinesterase. The latter plays a key role in the inactivation of ACh by splitting the molecule and as such helps determine the intrasynaptic concentrations of ACh. Unlike monoamine neurons, cholinergic neurons have no reuptake site for ACh; inactivation is brought about principally by enzymatic breakdown.

Two broad categories of receptor mediate the action of ACh, namely muscarinic and nicotinic. These receptors are widely distributed in the brain. Nicotinic receptors are ionotropic with rapid responses. Nicotine is the selective agonist at these receptors while nicotinic antagonists include tubocurarine and hexamethonium. Muscarinic receptors are metabotropic with slower responses, and are stimulated by muscarine and blocked by atropine. Acetylcholinesterase inhibition results in increases in ACh, with increased stimulation of both nicotinic and muscarinic receptors.

Nicotine withdrawal in the habituated individual results in a major decrease in the stimulation of nicotinic receptors with the emergence of significant withdrawal symptoms. Increased stimulation can be brought about by either re-instituting a nicotine agonist or by inhibiting acetylcholinesterase. Most subjects who discontinue nicotine consumption resume smoking within a short period to reduce the craving sensations. The use of an acetylcholinesterase inhibitor by increasing ACh levels would increase nicotinic and muscarinic receptor stimulation. By so doing it would help reduce craving for nicotine in the nicotine dependent individual. However use of such an inhibitor would also result in major muscarinic stimulation resulting in gastrointestinal and cardiovascular affects most notably nausea, vomiting and altered heart rate. Such side effects are unacceptable to most patients withdrawing from nicotine.

One previous patent, U.S. Pat. No. 5,480,651, describes the use of an acetycholinesterase inhibitor, which is physostigmine, in association with a muscarinic antagonist, which in its preferred embodiment is scopolamine, such that the antagonist action of scopolamine at muscarinic sites will inhibit the muscarinic agonism which leads to nausea and other unwanted side effects. However this invention is specific to the drugs mentioned.

U.S. Pat. No. 5,643,905 describes the use of galantamine in smoking cessation (also written as galanthamine). Experiments involving two subjects over 24 hours and employing a transdermal patch are described wherein craving is reduced or eliminated during the 24 hour period. This is suggestive of a role for galantamine in smoking cravings, but there is little supportive data, no long term data, and no information of how well or badly the galantamine is tolerated.

Another U.S. patent, U.S. Pat. No. 6,670,356, describes galantamine derivatives and indicates without any data that they may be useful in a number of conditions including smoking cessation.

Some studies have been carried out on the effects of antidepressants in smoking cessation, and as mentioned above, one such drug has been approved in the U.S. (buproprion, Zyban™). Its mechanism of action in smoking seems to be related to its effects on noradrenaline and dopamine re-uptake. The reversible MAO-A inhibitor moclobemide (Mannerix), which is available in Europe and Canada to treat major depression, has also demonstrated efficacy at 400 mg per day in a French placebo controlled trial for smoking cessation.

Clonidine, an alpha adrenoceptor which was approved originally for the treatment of hypertension and found to be useful subsequently for opiod withdrawal, has also been shown to be useful in smoking cessation in a number of studies. U.S. Pat. No. 4,788,189 discloses use of clonidine in association with IM injected tricyclic antidepressants to effect smoking cessation. However clonidine has numerous disadvantages as a drug for this application. Further, it is disclosed that the tricyclic antidepressant serves as a potentiator of the alpha 2 noradrenergic activity of clonidine by blocking reuptake of norepinephrine to further stimulate alpha 2 receptors. As such, the use of the tricyclic antidepressant is specific to their use as potentiators of norepinephrine inhibition.

Galantamine, discussed above, is the active ingredient in the drug Reminyl™ approved for treatment of mild to moderate Alzheimer's disease. The normal starting dose is two 4 mg tablets daily with gradual titration up to a maximum of 24 mg daily. Reminyl™ use and titration upwards is associated with nausea and vomiting in a proportion of patients.

By way of background, current therapy for Alzheimer's disease (AD) largely depends on acetylcholinesterases which raise brain levels of acetylcholine at both nicotinic and muscarinic acetylcholine receptors. Three such drugs are marketed in the US, including galantamine, rivastigmine and donepazil. These drugs result in acetylcholine binding to muscarinic sites and to varying degrees this results in nausea which is the main rate limiting step in terms of dosing. This is particularly significant for galantamine and rivastigmine. Johnson & Johnson, the company responsible for marketing galantamine under the brand name Reminyl™, recognizes the tolerability problems associated with this drug, and a recent patent application by Johnson & Johnson provides for a slower titration regimen of galantamine that is claimed to result in improved tolerability (US Patent Application 2003/0139391). Nevertheless significant amounts of GI side effects remain, especially at higher doses, even with the altered titration proposed. In addition, this slower treatment approach also suffers the disadvantage, as does the current titration regimen, that patients cannot be started on a therapeutic dose of galantamine.

Unlike other acetylcholinesterase inhibitors including rivastigmine and donepazil, galantamine acts as an allosteric modulator of the nicotinic acetylcholine receptor. According to Samochocki et al (J Pharmacol Exp Ther. 2003 June; 305(3):1024-36) galantamine is somewhat better in efficacy compared to the other two drugs and a recent head to head comparison, wherein donepazil showed that galantamine had significant advantages in terms of effect on cognition. This effect is apparently not related to the relative pharmacological action of these drugs as acetylcholinesterase inhibitors as galantamine is inferior to the other drugs in this regard. The authors conclude that the therapeutic benefit of galantamine is largely mediated through its effects as an allosteric modulator of the nicotinic receptor. However galantamine also seems to have other effects which may be beneficial in Alzheimer's disease. Recent in-vitro studies show that galantamine has a neuroprotective effect on neuroblastoma cell lines and acts to inhibit apoptosis induced by beta-amyloid and thapsigargin in a concentration dependent manner. (Esperanza, Arias et al. 2004, Neuropharmacology, Vol 46, 1, 103-114).

It is reasonable to conclude that a higher concentration of galantamine would have a more beneficial effect on the progression of AD, and may be able to extend the time period before return to baseline cognitive decline. At present, the usefulness of galantamine is limited by virtue of its tolerability. The drug is normally titrated up from a starting dose of 8 mg. While, the maximum amount approved or used is 24 mg, in the case of around 50% of patients it is necessary to reduce this maximum dose back to 16 mg/day or discontinue treatment. Thus any method by which galantamine could be made more tolerable would have beneficial effects on Alzheimer's patients and would also permit a wider population of Alzheimer's patients to be treated effectively with galantamine.

Despite these approaches, there remains a need for new treatments for nicotine dependence and dementia, such as AD.

### BRIEF SUMMARY OF THE INVENTION

The present invention generally relates to the use of acetylcholinesterase inhibitors in combination with tricyclic antidepressants with anti-muscarinic properties. In accordance with the present invention, it has been unexpectedly found that the combination acts synergistically to improve the use of acetylcholinesterase inhibitors to allow for higher doses and greater tolerability. Further, the tricyclic antidepressant is able to exert independent mechanisms of action.

As such, in one aspect of the invention, a method for the treatment of nicotine dependence is provided. The method comprises administering an effective amount of an acetylcholinesterase inhibitor and a tricyclic antidepressant with anti-muscarinic properties to a subject in need or desirous thereof.

In one embodiment, the treatment of nicotine dependence substantially effects or maintains smoking cessation. In another embodiment, the tricyclic antidepressant is effective to treat or mitigate at least one withdrawal symptom of said nicotine dependence to thereby effectuate said treatment of nicotine dependence. In yet another embodiment, the tricyclic antidepressant is effective to mitigate gastrointestinal side effects of said acetylcholinesterase inhibitor to thereby increase tolerance of said composition by said subject during said administration.

In one preferred embodiment, the acetylcholinesterase inhibitor is galantamine or a pharmaceutically acceptable salt thereof, and said tricyclic antidepressant is trimipramine or a pharmaceutically acceptable salt thereof.

In another aspect of the invention, a method of mitigating the gastrointestinal side effects of an acetylcholinesterase inhibitor is provided, The method comprises administering a acetylcholinesterase inhibitor to a subject in combination with an amount of a tricyclic antidepressant effective to mitigate the gastrointestinal side effects of said acetylcholinesterase inhibitor, as compared to the gastrointestinal side effects of said acetylcholinesterase inhibitor when administered to a subject in the absence of said tricyclic antidepressant. Again, in one embodiment, the acetylcholinesterase inhibitor is galantamine or a pharmaceutically acceptable salt thereof, and said tricyclic antidepressant is trimipramine or a pharmaceutically acceptable salt thereof.

In yet another aspect of the invention, a method for the treatment of Alzheimer's Disease and other vascular dementias is provided. The method comprises administering an effective amount of an acetylcholinesterase inhibitor and a tricyclic antidepressant with anti-muscarinic properties to a subject in need thereof.

Yet another aspect of the invention is directed to a pharmaceutical composition comprising an acetylcholinesterase inhibitor and a tricyclic antidepressant.

Yet another aspect of the invention is directed to a kit comprising galantamine and trimipramine tablets packed in a blister pack, optionally including an information leaflet including conditions of use.

Further features and advantages of the invention, as well as operation of various embodiments of the invention, are described in detail below with reference to the examples. It is noted that the invention is not limited to the specific embodiments described herein. Such embodiments are presented herein for illustrative purposes only. Additional embodiments will be apparent to persons skilled in the relevant art(s) based on the teachings contained herein.

### DETAILED DESCRIPTION OF THE INVENTION

In accordance with the present invention, it has been unexpectedly found that the administration of an acetylcholinesterase inhibitor in combination with a tricyclic antidepressant having anti-muscarinic properties provides a highly effective and well tolerated treatment for nicotine dependence and dementias, such as Alzheimer's disease (AD). In one aspect, it is effective in the treatment of nicotine dependence, as well as in the treatment and mitigation of nicotine withdrawal symptoms and in the effectuation of smoking cessation. In another aspect, it is effective in the treatment and mitigation of dementias such as Alzheimer's disease.

While not being bound by any one mechanisms of action, it is believed that the tricyclic antidepressant exerts independent mechanisms of action in the treatment of nicotine dependence and dementia, as well as being capable of mitigating the gastrointestinal side effects of the acetylcholinesterase inhibitor. As such, the bio-tolerance of acetylcholinesterase inhibitors is improved, thereby allowing for higher dosages and/or less gradual dosage titrations. In this regard, the tricyclic antidepressant exerts anti-muscarinic effects, as well as blocking re-uptake of both noradrenaline and serotonin.

As such, the present invention generally relates to methods and compositions for treating nicotine dependence, treating or mitigating withdrawal symptoms associated with nicotine dependence, or effectuating smoking cessation. The methods and compositions of the invention also relate to the mitigation of the gastrointestinal side effects of acetylcholinesterase inhibitors. Further, the methods and compositions of the invention also relate to the treatment and mitigation of dementias, such as Alzheimer's disease.

I. Compounds of the Invention

The acetylcholinesterase compounds of the invention include any known pharmaceutically acceptable acetylcholinesterase inhibitors. Preferred acetylcholinesterase inhibitors include physostigmine, neostigmine, rivastigmine, galantamine, donepazil, and pharmaceutically acceptable salts, racemates and isomers thereof. In a particularly preferred embodiment, the acetylcholinesterase inhibitor is galantamine, or a pharmaceutically acceptable salt thereof. Derivatives of galantamine are also contemplated as within the scope of the invention, as are known in the art. The chemical structure of galantamine is shown below.

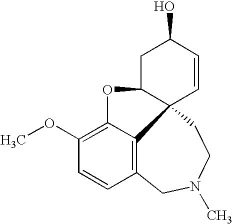

Galantamine is an acetylcholinesterase inhibitor that has been used to reverse the muscular effects of gallamine and tubocurarine, and has been approved as a treatment for Alzheimer's disease, and studied as a treatment for other central nervous system disorders. Galantamine may be obtained, for example, by isolation from the caucasian snowdrops Galanthus woronowi Vel., Amaryllidaceae, or by chemical synthesis, as known in the art.

The tricyclic antidepressant compounds of the invention include any pharmaceutically acceptable tricyclic antidepressant known in the art. Generally, tricyclic antidepressants (TCAs) include a group of chemically related analog compounds derived from imipramine. TCAs vary in their potency and monoamine selectively, but generally produce a reduction in the firing rate of neurons containing norepineprhrine. Exemplary TCAs useful in the context of the present invention include: amitriptyline, amoxapine, clomipramine, desipramine, doxepin, dothiepin, imipramine, nortriptyline, protriptyline, trimipramine, and pharmaceutically acceptable salts thereof. Particularly preferred is trimipramine and pharmaceutically acceptable salts thereof (e.g., trimipramine maleate). The chemical structure of trimipramine is shown below.

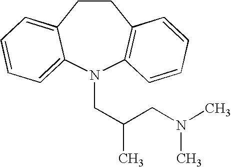

Also falling within the scope of the present invention are the in vivo metabolic products of the compounds described herein. Such products may result for example from the oxidation, reduction, hydrolysis, amidation, esterification and the like of the administered compound, primarily due to enzymatic processes. Accordingly, the invention includes compounds produced by a process comprising contacting a compound of the invention with a mammalian tissue or a mammal for a period of time sufficient to yield a metabolic product thereof. Such products typically are identified by preparing a radio-labeled (e.g. C¹⁴ or H³) compound of the invention, administering it in a detectable dose (e.g., greater than about 0.5 mg/kg) to a mammal such as rat, mouse, guinea pig, monkey, or to man, allowing sufficient time for metabolism to occur (typically about 30 seconds to 30 hours), and isolating its conversion products from urine, blood, tumor, or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds of the invention even if they possess no biological activity of their own.

II. Methods of the Invention

In accordance with one aspect of the present invention, it has been discovered that acetylcholinesterase inhibitors, when administered in combination with TCAs with anti-muscarinic properties, are a particularly effective treatment for nicotine dependence. It has also been found that the acetylcholinesterase inhibitors, in combination with TCAs, are particularly effective to treat and/or mitigate withdrawal symptoms associated with nicotine dependence.

In another aspect of the invention, it has been discovered that TCAs with anti-muscarinic properties, when administered in combination with acetylcholinesterase inhibitors, are effective to mitigate the gastrointestinal side effects of the acetylcholinesterase inhibitors.

In yet another aspect, it has been discovered that acetylcholinesterase inhibitors, in combination with TCAs, are particularly effective to treat and/or mitigate dementia, such as Alzheimer's disease (AD).

By way of example, the combination of an acetylcholinesterase inhibitor such as galantamine and an tricyclic antidepressant with anti-muscarinic activity such as trimipramine will allow the galantamine to exert its cholinergic effects solely through the nicotinic receptor system. Furthermore, as mentioned above, trimipramine blocks the re-uptake of both noradrenaline and serotonin. Noradrenaline is mainly produced in the nucleus locus coeruleus and regulates levels of arousal and vigilance. Serotonin is produced in the brain stem raphe neuronal network and is known to have several functions including the regulation of appetite. Alteration in levels of arousal and alteration of appetite are key components in the nicotine withdrawal syndrome. Trimipramine will not only reduce the anti-muscarinic side-effects of galantamine but will also help alleviate two major features of the withdrawal syndrome.

In addition to positive reinforcement (e.g., smoking satisfaction), withdrawal and craving; several secondary effects of nicotine and tobacco smoke use contribute to both the maintenance of smoking and smoking relapse including mood modulation (e.g., reduction of negative affect, stress reduction and cognitive enhancement). Nicotine withdrawal is characterized by feelings of stress, irritability, and anger. Nicotine addiction is maintained by reversal of deprivation induced stress and negative mood observed between cigarettes. It is known that nicotine enhances cognitive function and investigations are underway in attention deficit, including Alzheimer's disease (AD) into its possible use in this area. Degraded attention and cognition following a period of nicotine deprivation can be a strong motivating factor to smoke to reverse such deficits. Without intending to be limited by theory, it is believed that addressing these factors will play a key role in any combination therapy approach to smoking cessation.

While the use of acetylcholinesterase inhibitors such as galantamine in the treatment of nicotine dependence is generally known, it was unexpectedly discovered in accordance with the present invention that such use is particularly effective in combination with a TCA with anti-muscarinic properties. Without intending to be limited by theory, the acetylcholinesterase inhibitor and the TCA with anti-muscarinic properties are believed to act synergistically to achieve an overall efficacy that is unexpected. Not only do the acetylcholinesterase inhibitor and the TCA exert independent mechanisms of action to treat nicotine dependence and/or withdrawal symptoms associated therewith, but it is believed that the TCA acts synergistically to mitigate gastrointestinal side effects of the acetylcholinesterase, as known side effect. In this manner, higher doses of the acetylcholinesterase inhibitor are available, if desired. Alternatively, less gradual titration to higher doses of the acetylcholinesterase may be required to achieve sufficient subject tolerance.

As such, one embodiment relates to methods for treating nicotine dependence. The method generally includes administering an effective amount of an acetylcholinesterase inhibitor and a tricyclic antidepressant to a subject in need or desirous thereof. In another aspect, a method for maintaining the non-smoking status of a subject is provided (e.g., maintenance of the mitigation of nicotine dependence).

A further embodiment of the invention relates to methods for maintaining smoking abstinence following completion of the main course of treatment. This method comprises the use of the same or a lower dose combination of the invention which is taken for some months following the end of the main course of therapy as envisaged herein. The effect of the lower dose is to counteract the on-going nicotine cravings. Additionally the composition of the invention in the doses described herein and the lower maintenance dose as contemplated herein may be employed by smokers in situations where smoking is not permitted, for example during a long haul flight or a stay in hospital or in countries or cities where smoking in public is not permitted.

A yet further embodiment of the invention relates to methods for improving the success rate in subjects taking tobacco vaccines. Such vaccines employ nicotine binding antibodies which are unable to cross the blood brain barrier. However they do not affect the craving for nicotine. Accordingly the compositions of the invention may be employed in combination with such a vaccine to provide a more effective therapy and maintenance of non-smoking status.

In a further application of the invention a method of achieving or maintaining smoking cessation in schizophrenic patients is provided. The smoking rate for patients with schizophrenia reaches as high as 90% in clinical samples (Dudas M M and T P George. Non-nicotine pharmacotherapies for nicotine dependence. 2005; 6(3): 158-72). Schizophrenia is associated with a number of other serious risks to health most significantly being increased weight gain, increased risk of Type II diabetes and metabolic syndrome and increased risk of coronary heart disease. Whether the increased risk of diabetes results from the underlying condition or in part from use of certain anti-psychotic drugs is unclear at this time. However it is evident that smoking is a serious health risk factor in such patients and there is an urgent need for a pharmacotherapy which can assist in quitting.

Nicotine use by schizophrenics seems to be a form of self medication in that the cognitive enhancing properties of nicotine have been established in other populations. A number of studies have considered the role of cholinergic systems in schizophrenia and have carried out controlled trials of the augmentation of therapy with acetylcholinesterase inhibitors most especially donepazil. These studies were reviewed by Friedman in 2004. (Friedman J I. Psychopharmacology (Berl.). 2004 June; 174(1); 45-53.) Although some open label studies demonstrate modest cognitive improvements of schizophrenic patients treated with donepazil, data from a blinded placebo controlled study demonstrate no effect. However there is some limited data suggesting that galantamine may have beneficial effects in schizophrenia (Allen et al. 2002. Galantamine for Treatment resistant schizophrenia. Am J Psychiatry 159: 1244-1245).

As such, in another embodiment, the present invention provides for a method for the effective treatment of nicotine dependence in schizophrenic patients. It has been unexpectedly found that administering the combination of galantamine with trimipramine resulted in a significant reduction in both craving and use of tobacco by schizophrenia patients (medicated with anti-psychotics). The use of the adjunct medication was well tolerated and did not have any adverse effects on the use of the antipsychotic drug on positive or negative symptoms of the disease.

In another aspect, the combination of the invention may be administered in conjunction with anti-psychotic medication as an augmentation strategy for treatment of psychoses and in particular to act as a cognitive enhancer in schizophrenic patients. The use of acetylcholinesterase inhibitors as an augmentation medication in schizophrenia has been described in U.S. Pat. No. 5,633,238. The invention herein provides an improvement on the teachings of that Patent in that higher doses and greater tolerability of the combination of the invention will provide improved therapeutic benefit to the schizophrenic patient in comparison to the use of an acetylcholinesterase inhibitor alone.

For instance, the combination of the invention may be administered in conjunction with benzodiazepines to counteract the typical sedative and the hypnotic effects of benzodiazepines. See, e.g., U.S. Pat. No. 5,633,238, which is herein incorporated by reference in its entirety. Subjects may be treated with amounts of benzodiazepine which are sufficient with respect to the desired effect on their condition, such amounts being established, e.g., in accordance with normal principles in benzodiazepine therapy, that is, by monitoring the symptoms of the disease to be treated and thereby establish an individual dosage which is effective. However, due to the use, according to the invention, of a acetylcholinesterase inhibitor in combination with a TCA to counteract the sedative or hypnotic effects, the limitation on the dosages previously imposed due to acetylcholinesterase and benzodiazepine side effects, is no longer necessary, and thus, a more efficient treatment with the benzodiazepines may be obtained. As such, in another embodiment, a method for treating schizophrenia is provided, said method comprising administering an acetylcholinesterase inhibitor in combination with a TCA in accordance with the present invention to a subject in need thereof, wherein said subject is a patient undergoing treatment with benzodiazepines.

The diseases treated with benzodiazepines constitute a broad spectrum of diseases because of the many effects of the benzodiazepines. Diseases where the sedative or hypnotic effects of the benzodiazepines are undesirable are diseases in connection with which the principle of the present invention is particularly important. Especially the treatment of the following diseases: anxiety, anxiety neurosis, anxiety reactions, panic reactions, schizophrenia, affective type schizophrenia, borderline psychosis, agitated endogenous depressions, hyperactivity in children, and muscle spasms, may benefit from the use of both a benzodiazepine and a cholinesterase inhibitor in accordance with the principle of the invention, as these diseases are known to require high dosages of benzodiazepine in order to obtain the benefit of the benzodiazepine therapy, the high dosages, on the other hand, incurring the above-mentioned severe disadvantages due to the sedative and hypnotic effects if no administration of cholinesterase inhibitor is performed in connection with the benzodiazepine treatment.

Schizophrenia and affective type schizophrenia, and schizoaffective type of schizophrenia are conditions in which benzodiazepine therapy is useful, as well as adjunct therapy with the combination of the invention. However, according to the present invention these conditions may also be treated with a combination of the invention alone.

The combination of the invention will not necessarily be given at the same time as the benzodiazepine. Thus, e.g., if, after some time of administration of a benzodiazepine as the sole or main medication, the sedative or hypnotic effects of the benzodiazepine has become a clinical problem, a combination of the invention may be administered to counteract the sedative or hypnotic effects either in addition to the benzodiazepine or alone if the treatment with benzodiazepine has been stopped temporarily. From this it will be understood that the combination of the invention may also be used in the treatment of sedative or the hypnotic effects resulting from an overdose of benzodiazepine.

In a further aspect, it has been discovered that the tolerability of the acetylcholinesterase inhibitor can be greatly improved by administering the inhibitor in combination with the TCA. At present, the effective use of a number of acetylcholinesterase inhibitors is limited by their gastrointestinal side effect profiles. As such, in one embodiment, a method for reducing gastrointestinal side effects associated with acetylcholinesterase inhibitors is provided. In another embodiment, a method for treating dementia such as Alzheimer's disease or vascular dementia (where affective symptomatology can be a significant issue) is provided comprising administering to a subject in need thereof, an acetylcholinesterase inhibitor, such as galantamine, in combination with a TCA, such as trimipramine. In accordance with the present invention, it has been found that the combination is exceptionally well tolerated in subjects, which provides for an improvement in the use of, e.g., galantamine for the treatment of Alzheimer's disease and other dementias.

By way of example, it has been unexpectedly found that the combination, e.g., galantamine and trimipramine, is extremely well tolerated and is devoid of the nausea which is characteristic of galantamine therapy in the treatment of Alzheimer's disease, and other dementias such as vascular dementia, Lewy body dementia or dementia associated with neurological diseases. The invention further provides a way wherein higher doses of the acetylcholinesterase inhibitor (e.g., galantamine) may be given to Alzheimer's patients without the rate limiting side effects of current galantamine therapy and with corresponding greater benefits in terms of slowing cognitive decline than is currently possible by employing galantamine on its own.

Most patients with multi-infarct dementia experience depressive symptoms and the use of the TCA is believed to therefore be of additional benefit. Without intending to be limited by theory, the presence of the TCA, which blocks noradrenaline reuptake as well as serotonin reuptake in addition to the anti-muscarinic activity, adds synergistic benefit to the composition. Noradrenaline is mainly produced in the nucleus locus coeruleus and regulates levels of arousal and vigilance. Thus the presence of this antidepressant in the medication not only improves the tolerability of galantamine for Alzheimer's patients but also assist in sleep. Night-time agitation, confusion, and wandering by Alzheimer's patients is a major concern for care givers. A treatment of these symptoms can therefore have significant pharmacoeconomic benefits during the care of the Alzheimer's patient.

According to the methods of the invention, the compound(s) may be administered to the subject via any drug delivery route known in the art. Specific exemplary administration routes include peripheral and central routes such as oral, ocular, rectal, buccal, topical, nasal, ophthalmic, subcutaneous, intramuscular, intraveneous (bolus and infusion), intracerebral, transdermal, and pulmonary. In a preferred embodiment, the composition is administered orally via tablet or a combination of tablets.

The acetylcholinesterase inhibitor (e.g., galantamine) and the tricyclic antidepressant (e.g., trimipramine) may be combined in any manner known in the art such as a unitary dosage form, or in separate dosage forms intended for simultaneous or sequential administration to a patient in need of treatment. When administered sequentially, the combination may be administered in two or more administrations. In an alternative embodiment, it is possible to administer one or more compounds of the present invention and one or more additional active ingredients by different routes.

According to the methods of the invention, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

The term “effective amount”, as used herein, refers to an amount of a pharmaceutical agent to treat, ameliorate, or mitigate the identified disease or condition, or to exhibit a detectable therapeutic or inhibitory effect. The effect can be detected by any means known in the art. The precise effective amount for a subject will depend upon the subject's body weight, size, and health; the nature and extent of the condition; and the agent or combination of agents selected for administration. Effective amounts for a given situation can be determined by routine experimentation that is within the skill and judgment of the clinician.

More particularly, preferred effective amounts of the acetylcholinesterase inhibitor of the present invention include administration at doses that vary from about 8 mg per day to about 50 mg per day, preferably 12 mg per day to about 32 mg per day, preferably about 16 mg per day to about 32 mg per day, or preferably about 16 mg per day to about 24 mg per day, administered in single or divided doses, depending upon the route of administration. Guidance as to particular dosages and methods of delivery is provided in the literature and is generally available to practitioners in the art. Recommended dosages for the TCA as employed in practice the present invention are about 10 mg per day to about 100 mg per day, preferably 40 mg per day to about 100 mg per day, or preferably 60 mg per day to about 100 mg per day, in single or divided doses, depending on patient response. The compounds may be administered in one, two, three, four, five, etc. doses per day, as needed. Higher or lower doses may be employed for if desired, as recognized by those skilled in the art.

Further, it may be desired to use multiple dosing schemes in connection with the treatment or mitigation of nicotine dependence or nicotine withdrawal symptoms. On the other hand, it may be desirable to use single dosing schemes (or two doses) with the treatment or mitigation of dementias such as Alzheimer's to simplify the dosing regime.

By way of example, at present patients with Alzheimer's Disease ( for which galantamine is approved) are titrated upwards from 8 mg to 24 mg. The present invention provides a means whereby patients may be started at a higher dose and a larger number maintained on the maximum dose of 24 mg. Thus the fixed drug combination for the above purposes may be formulated in such a manner as to deliver a starting daily dose in the range of 12-16 mg of galantamine daily administered in divided doses.

In a further embodiment, the invention provides a twice a day dosing employing a composition of 12 mg galantamine per tablet plus an appropriate amount of trimipramine which may be in the range of 40-100 mg, in single or divided doses as needed.

In a further embodiment of the invention, the daily dose of galantamine may be safely increased beyond that currently employed i.e., in levels greater than 24 mg per day. The higher levels of galantamine possible under the invention provide for a significant improvement in the slowing of cognitive decline.

In yet a further embodiment of the invention the daily dose of galantamine and trimipramine may be delivered by means of a sustained release formulation wherein the daily dose of galantamine given is in the range of about 16 mg-32 mg daily, in single or divided doses.

The exact dosage will be determined by the practitioner, in light of factors related to the subject that requires treatment and the form of compound used (e.g., the salt form). Dosage and administration are adjusted to provide sufficient levels of the active agent(s) or to maintain the desired effect. Other factors which may be taken into account include the severity of the disease state, general health of the subject, age and weight of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

III. Pharmaceutical Compositions of the Invention

In yet another aspect of the invention, pharmaceutical compositions useful in the methods of the invention are provided. The pharmaceutical compositions of the invention may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8.0.

More particularly, the pharmaceutical compositions of the invention comprise a therapeutically effective amount of the compound(s) or a pharmaceutically acceptable salt thereof, together with one or more pharmaceutically acceptable excipients. For instance, when the pharmaceutical composition is formulated as an oral tablet, the composition preferably comprises from about 1.0 mg to about 100 mg of the compound(s). As discussed above, the exact amount of compound(s) may vary. In one embodiment, the pharmaceutical composition comprises a single active ingredient, i.e., only the acetylcholinesterase inhibitor or TCA is included in the pharmaceutical composition, e.g., the composition consists essentially of the acetylcholinesterase inhibitor or TCA or a pharmaceutically acceptable salt thereof. In another embodiment, the pharmaceutical composition comprises a combination of active ingredients, i.e., two or more active ingredients, e.g., the acetylcholinesterase inhibitor and the TCA.

Alternatively, the pharmaceutical compositions of the invention may comprise an additional active ingredients including additional therapeutic agents useful in the treatment of nicotine dependence, Alzheimer's disease or dementia. Therapeutic amounts of additional agents are generally known in the art or may be determined by the skilled clinician.

Formulations of the present invention, e.g., for parenteral or oral administration, are most typically solids, liquid solutions, emulsions or suspensions, while inhaleable formulations for pulmonary administration are generally liquids or powders, with powder formulations being generally preferred. Alternative pharmaceutical compositions of the invention may be formulated as syrups, creams, ointments, tablets, and the like.

The term “pharmaceutically acceptable excipient” refers to an excipient for administration of a pharmaceutical agent, such as the compounds of the present invention. The term refers to any pharmaceutical excipient that may be administered without undue toxicity. Pharmaceutically acceptable excipients are determined in part by the particular composition being administered, as well as by the particular method used to administer the composition. Accordingly, there exists a wide variety of suitable formulations of pharmaceutical compositions of the present invention (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions of the invention may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed. Tablets may be formulated as controlled release drugs using techniques known in the art so as to provide once a day dosing within the ranges as specified herein. In another embodiment, tablets may be formulated in an immediate release form but in higher doses than those previously discussed provide an alternative form of once a day dosing.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions of the invention may be formulated as suspensions comprising a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

IV. Kits and Blister Packs

In another aspect of the invention, the pharmaceutical compositions of the invention may be provided as kits, optionally including informational and/or instructional leaflets. The kits may comprise a blister pack including tablets, capsules, etc. Each tablet, capsule, etc. may include a single active ingredient, or may include a combination of active ingredients, as described above. For instance, each tablet, capsule, etc. may include the acytlcholinesterase inhibitor as a single active ingredient, the tricyclic antidepressant as a single active ingredient, or may include a combination of the two.

Dosages within the tablets, capsules, etc, may be such that one, two, three, four, etc. doses are taken per day, as recognized by those of skill in the art. By way of example, the use of multiple dosing throughout the day may provide a substitute behavioral pattern which assists in combating the well established behavioral patterns associated with nicotine addiction. Additionally the use of individual tablets for the individual active ingredients permits flexibility on the part of the user to manage any unwanted side effects which might differ between individuals, and/or to provide gastrointestinal side effect relief (e.g., nausea) when needed.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof. The examples illustrate the preparation of certain compounds of the invention, and the testing of these compounds. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

### Example 1

The following describes a double-blind placebo controlled trial on 107 subjects, carried out at one centre in Cork, Ireland between November 2004 and May 2005.

Healthy (physically and mentally fit) male and female volunteers (aged 18-65) were recruited. Volunteers were smoking at least 15 cigarettes per day for the past 12 months and had a demonstrated desire to quit.

Galantamine was administered to each volunteer (4 mg tds) and trimipramine (20 mg tds). Provision was made to reduce either galantamine to 4 mg bd in case of intolerable nausea/GI upset and/or trimipramine to 20 mg bd if intolerable sedation.

On day 0 cigarette smoking was allowed and on the evening of day 0, trimipramine (20 mg) or placebo is taken alone. Volunteers were expected to be abstinent after that dose and from day 1 onwards.

Between week 7 and week 8 the dose of the combination treatments and placebo were decreased by one capsule for 2 days, a further capsule for 2 days and if necessary by one tablet for a further 2 days to ensure gradual withdrawal of medication.

Safety was assessed by physical examination and medical history at days 0 and physical examination at week 8 and medical history at the initial screening visits. HR and BP were tested at each visit. Adverse event monitoring system was in place.

Efficacy was measured by self-reporting of smoking abstinence confirmed by an expired breath CO concentration of 10 ppm or less.

Primary efficacy variables were ; continuous abstinence to week 7. Abstinence criteria were defined as self-report of no smoking and all study measurements of expired CO concentrations of 10 ppm or less at every visit.

Secondary efficacy variables were; abstinence rates at each visit, time to first relapse, number of cigarettes smoked, relapse between weeks 7 and 8 and weight change.

To be considered abstinent, subjects participating in the study were required to meet all two of the following criteria during the 7 weeks of treatment;

(a) self-report of no smoking

(b) All study measurements of expired carbon monoxide (CO)≦10 ppm.

Subjects with missing data were counted as smokers (non-abstinent) at the intervals at which data was missing.

Results

The combination of 4 mg three times per day and trimipramine 20 mg, 3 times per day was very well tolerated with very few GI symptoms being reported. Of 107 patients enrolled, none were withdrawn due to GI side effects. The following is an extract from the full data set which represents an interim safety analysis of a subset of subjects.

22 subjects included as volunteers were randomised to receive either galantamine (GAL) 4 mg tds plus trimipramine (TRIM) 20 mg tds or placebo. 12 (54.5%) were female, 10 (45.5%) male. Mean age was 37 years (range 22-56). Subjects were healthy with no concurrent confounding medical conditions, in particular gastro-intestinal conditions (dyspepsia, irritable bowel), cardiac disease, renal or hepatic impairment. Subjects were treated for varying durations due to study withdrawals ranging from 1 day (n=1) to a maximum of 50 days. Mean duration was 27 days. Out of the 22 subjects a minimum of 12, maximum of 14 may be considered as having been randomised to active (GAL/TRIM) medication. There was no report or observation of any gastro-intestinal adverse event

This compares to information we have from clinicians active in the Alzheimer's field that 15-20% of their patients cannot tolerate the starting dose of 8 mg (75% of our starting dose) and a further 10-15% cannot get past 16 mg per day. Around 50% of Alzheimer's patients cannot reach 24 mg of galantamine daily.

Our data is strengthened by the fact that Alzheimer's patients are less responsive to the nausea inducing effects of galantamine than are normal healthy volunteers due to existing damage to the cholinergic system in the former.

The data were analyzed as to the other main end-point of smoking cessation. There was a high drop out rate which is normal for studies in this area (e.g., Zyban has an efficacy of only around 30%). On an intent-to-treat basis, a statistically significant (p<0.05) number of patients on active drug were found to be abstinent at the end of the treatment period.

### Example 2

A 60 year old patient with a long history of chronic schizophrenia had been smoking approximately 40 cigarettes each day since his mid teens. He had been symptomatically stable for 1 year and his antipsychotic medication remained unaltered over this period. He was prescribed 12 mg daily of galantamine in combination with 60 mg of trimipramine and given advice to stop smoking. His motivation was assessed as moderate. He stopped smoking and has remained nicotine free for 3 months. The galantamine and trimipramine have been continued as he describes a definite improvement in general well being whilst taking the drugs.

1. A method for the treatment or mitigation of nicotine dependence, said method comprising administering an effective amount of an acetylcholinesterase inhibitor and a tricyclic antidepressant with anti-muscarinic properties to a subject in need or desirous thereof.

2. The method of claim 1, wherein said treatment or mitigation of nicotine dependence substantially effects or maintains smoking cessation.

3. The method of claim 1, wherein said tricyclic antidepressant is effective to treat or mitigate at least one withdrawal symptom of said nicotine dependence to thereby effectuate said treatment of nicotine dependence.

4. The method of claim 1, wherein said tricyclic antidepressant is effective to mitigate gastrointestinal side effects of said acetylcholinesterase inhibitor to thereby increase tolerance of said composition by said subject during said administration.

5. The method of claim 1, wherein the acetylcholinesterase inhibitor is selected from the group consisting of: physostigmine, neostigmine, rivastigmine, galantamine, donepazil, or pharmaceutically acceptable salts, racemates or isomers thereof.

6. The method of claim 1, wherein the acetylcholinesterase inhibitor is galantamine or a pharmaceutically acceptable salt thereof.

7. The method of claim 1, wherein said tricyclic antidepressant is selected from the group consisting of: amitriptyline, amoxapine, clomipramine, desipramine, doxepin, dothiepin, imipramine, nortriptyline, protriptyline, trimipramine, and pharmaceutically acceptable salts thereof.

8. The method of claim 1, wherein the tricyclic antidepressant is trimipramine or a pharmaceutically acceptable salt thereof.

9. The method of claim 1, wherein said acetylcholinesterase inhibitor is galantamine or a pharmaceutically acceptable salt thereof, and said tricyclic antidepressant is trimipramine or a pharmaceutically acceptable salt thereof.

10. The method of claim 1, wherein between about 8 mg/day to about 50 mg/day of said acetylcholinesterase inhibitor is administered to said subject, and between about 40 mg/day to about 100 mg/day of said tricyclic antidepressant is administered to said subject.

11. The method of claim 1, wherein between about 12 mgs and about 32 mgs of said acetylcholinesterase inhibitor is administered to said subject daily in single or divided doses, and between about 60 mgs and about 100 mgs of said tricyclic antidepressant is administered to said subject daily in single or divided doses.

12. The method of claim 1, wherein said acetylcholinesterase inhibitor is administered to said subject once, twice, three times, or four times daily.

13. The method of claim 1, wherein said tricyclic antidepressant is administered to said subject once, twice, three times, or four times daily.

14. The method of claim 1, wherein said subject is suffering from schizophrenia.

15. A method of claim 1, wherein said administration comprises self-dosing throughout the day by said subject to thereby potentiate the treatment of nicotine dependence through provision of substitute behavioral patterns associated with nicotine use.

16. A method of mitigating the gastrointestinal side effects of an acetylcholinesterase inhibitor, said method comprising administering a acetylcholinesterase inhibitor to a subject in combination with an amount of a tricyclic antidepressant effective to mitigate the gastrointestinal side effects of said acetylcholinesterase inhibitor, as compared to the gastrointestinal side effects of said acetylcholinesterase inhibitor when administered to a subject in the absence of said tricyclic antidepressant.

17. The method of claim 16, wherein the acetylcholinesterase inhibitor is selected from the group consisting of: physostigmine, neostigmine, rivastigmine, galantamine, donepazil, or pharmaceutically acceptable salts, racemates or isomers thereof.

18. The method of claim 16, wherein the acetylcholinesterase inhibitor is galantamine or a pharmaceutically acceptable salt thereof.

19. The method of claim 16, wherein said tricyclic antidepressant is selected from the group consisting of: amitriptyline, amoxapine, clomipramine, desipramine, doxepin, dothiepin, imipramine, nortriptyline, protriptyline, trimipramine, and pharmaceutically acceptable salts thereof.

20. The method of claim 16, wherein the tricyclic antidepressant is trimipramine or a pharmaceutically acceptable salt thereof.

21. The method of claim 16, wherein said acetylcholinesterase inhibitor is galantamine or a pharmaceutically acceptable salt thereof, and said tricyclic antidepressant is trimipramine or a pharmaceutically acceptable salt thereof.

22. A method for the treatment of Alzheimer's Disease and other vascular dementias, said method comprising administering an effective amount of an acetylcholinesterase inhibitor and a tricyclic antidepressant with anti-muscarinic properties to a subject in need thereof.

23. The method of claim 22, wherein the acetylcholinesterase inhibitor is selected from the group consisting of: physostigmine, neostigmine, rivastigmine, galantamine, donepazil, or pharmaceutically acceptable salts, racemates or isomers thereof.

24. The method of claim 22, wherein the acetylcholinesterase inhibitor is galantamine or a pharmaceutically acceptable salt thereof.

25. The method of claim 22, wherein said tricyclic antidepressant is selected from the group consisting of: amitriptyline, amoxapine, clomipramine, desipramine, doxepin, dothiepin, imipramine, nortriptyline, protriptyline, trimipramine, and pharmaceutically acceptable salts thereof.

26. The method of claim 22, wherein the tricyclic antidepressant is trimipramine or a pharmaceutically acceptable salt thereof.

27. The method of claim 22, wherein said acetylcholinesterase inhibitor is galantamine or a pharmaceutically acceptable salt thereof, and said tricyclic antidepressant is trimipramine or a pharmaceutically acceptable salt thereof.

28. The method of claim 22, wherein between about 8 mg/day to about 50 mg/day of said acetylcholinesterase inhibitor is administered to said subject, and between about 40 mg/day to about 100 mg/day of said tricyclic antidepressant is administered to said subject.

29. The method of claim 22, wherein between about 12 mgs and about 32 mgs of said acetylcholinesterase inhibitor is administered to said subject daily in single or divided doses, and between about 60 mgs and about 100 mgs of said tricyclic antidepressant is administered to said subject daily in single or divided doses.

30. The method of claim 22, wherein said acetylcholinesterase inhibitor is administered to said subject once, twice, three times, or four times daily.

31. The method of claim 22, wherein said tricyclic antidepressant is administered to said subject once, twice, three times, or four times daily.

32. A pharmaceutical composition comprising an acetylcholinesterase inhibitor and a tricyclic antidepressant with anti-muscarinic properties.

33. The pharmaceutical composition of claim 32, wherein said tricyclic antidepressant is present in an amount effective to mitigate gastrointestinal side effects of said acetylcholinesterase inhibitor when administered to a subject at doses sufficient to cause gastrointestinal side effects when administered in the absence of said tricyclic antidepressant.

34. The pharmaceutical composition of claim 32, wherein the acetylcholinesterase inhibitor is selected from the group consisting of: physostigmine, neostigmine, rivastigmine, galantamine, donepazil, or pharmaceutically acceptable salts, racemates or isomers thereof.

35. The pharmaceutical composition of claim 32, wherein the acetylcholinesterase inhibitor is galantamine or a pharmaceutically acceptable salt thereof.

36. The pharmaceutical composition of claim 32, wherein said tricyclic antidepressant is selected from the group consisting of: amitriptyline, amoxapine, clomipramine, desipramine, doxepin, dothiepin, imipramine, nortriptyline, protriptyline, trimipramine, and pharmaceutically acceptable salts thereof.

37. The pharmaceutical composition of claim 32, wherein the tricyclic antidepressant is trimipramine or a pharmaceutically acceptable salt thereof.

38. The pharmaceutical composition of claim 32, wherein said acetylcholinesterase inhibitor is galantamine or a pharmaceutically acceptable salt thereof, and said tricyclic antidepressant is trimipramine or a pharmaceutically acceptable salt thereof.

39. The pharmaceutical composition of claim 32, wherein said acetylcholinesterase inhibitor is present in an amount ranging from about 2 to about 50 mgs, and said tricyclic antidepressant is present in an amount ranging from about 10 to about 100 mgs.

40. A kit comprising galantamine tablets or capsules and trimipramine tablets or capsules packed individually in a blister pack.

41. The kit of claim 40, further comprising an information leaflet including conditions of use.
